(19) United States
(12) Patent Application Publication
(10) Pub. No.: US 2021/0017200 A1
(43) Pub. Date: Jan. 21, 2021

(54) Hydrocarbyl Tin Complex with Antitumor Activity and Preparation Method Thereof

(71) Applicant: Zhejiang Hongsheng Intellectual Property Operation Co., Ltd., hangzhou (CN)

(72) Inventors: Zhongda DUAN, Hangzhou (CN); Jiande JIN, hangzhou (CN)

(21) Appl. No.: 17/061,335

(22) Filed: Oct. 1, 2020

(30) Foreign Application Priority Data
Apr. 18, 2020 (CN) 202010308167.X

(51) Int. Cl. C07F 7/22; A61P 35/00

(52) U.S. Cl. CPC C07F 7/226; A61P 35/00

(57) ABSTRACT

Hydrocarbyl tin complexs of alkynyl phosphonic acid with antitumor activity and their preparation method are provided in present invention. Tests are conducted to evaluate activity, showing that the provided complexs have much stronger activity than cisplatin, especially for human colon cancer. The provided hydrocarbyl tin complexs can be potential candidate as a clinical antitumor drug.

### CROSS REFERENCE TO RELATED APPLICATION

The application claims the priority of the Chinese patent application filed on Apr. 18, 2020, with the application number of 202010308167.X and the invention title of “Hydrocarbyl Tin Complex with Antitumor Activity and Preparation Method Thereof”, the entire contents of which are incorporated herein by reference.

### TECHNICAL FIELD

The present invention relates to a hydrocarbyl tin complex with antitumor activity, and belongs to the field of pharmaceutical chemistry.

### BACKGROUND

The phosphonic (phosphoric) acid derivatives of hydrocarbyl tin have attracted great attention due to their strong insecticidal, bactericidal, herbicidal and other biological activities. There are some literatures about the study of hydrocarbyl tin complex of alkynyl phosphonic acid, but most of them focus on the synthesis and structure research stage, yet there are few researches on its pharmacological activities. Furthermore, most of them are intended for their bactericidal and acaricidal activities, yet few researches are conducted to find out their antitumor activities.

### SUMMARY OF THE INVENTION

The present invention provides a hydrocarbyl tin complex with antitumor activity and preparation method thereof, to explore new antitumor drugs.

On the basis of full investigation of the prior art, the present invention screens out new hydrocarbyl tin complex of alkynyl phosphonic acid with antitumor activity, so as to provide a new candidate for antitumor compound.

In order to achieve the above purpose, the technical solution of the present invention is realized as follows:

A hydrocarbyl tin complex with antitumor activity is provided, having the following structure:

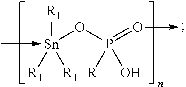

wherein, R1 is selected from n-butyl, t-butyl, phenyl, benzyl, p-chlorobenzyl and dimethylbenzyl;

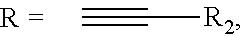

and R₂ is a randomly substituted alkyl, aryl and heteroaryl; n is an integer from 2.

Further, R₂ is a randomly substituted C₁-C₄ alkyl, C₆-C₁₀ aryl and C₆-C₁₀ heteroaryl;

Further, R₂ is a randomly substituted phenyl; and

the random substituent of R₂ is a halogen, such as F, Cl, Br, I; a C₁-C₈ alkyl, such as methyl, ethyl, propyl, t-butyl; a C₁-C₈ alkoxy, such as methoxy, ethoxy, propoxy, t-butoxy; nitryl, cyano group, trifluoromethyl; and the number of the substituents is 1, 2, 3, 4, or 5.

The above-mentioned tin complexes of alkynyl phosphonic acid are used for the preparation of antitumor drugs, which are specifically targeted for cervical cancer, breast cancer, lung adenocarcinoma, liver cancer, prostate cancer, colon cancer, etc.

The complexes of the present invention have the following beneficial effects:

In the present invention, the structure of the tin complexes of alkynyl phosphonic acid is modified, and several compounds with antitumor activity are screened out therefrom, which show much stronger activity than cisplatin, especially for human colon cancer, so it is a candidate to be a potential clinical anticancer drug. During the experiment, it is found that the change of the alkyl part (R₁) in the hydrocarbyl tin had a greater influence on the biological activity of the complexes, and the n-butyl has the best effect; and the kinds of substituents on the benzene ring also contribute a lot to its biological activity.

### DETAILED DESCRIPTION OF THE EMBODIMENTS

In order to make the purpose, technical solution and advantages of the present invention clearer, the representative embodiments of the present invention will be described in detail below, but the present invention is not limited thereto.

The present invention provides a hydrocarbyl tin complex with antitumor activity, having the following structure:

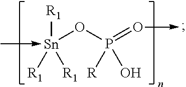

wherein, R₁ is selected from n-butyl, t-butyl, phenyl, benzyl, p-chlorobenzyl and dimethylbenzyl;

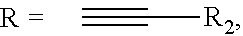

and R₂ is a randomly substituted alkyl, aryl and heteroaryl; n is an integer from 2.

In some embodiments, R₁ can be n-butyl, t-butyl, phenyl, benzyl; R₂ can be a randomly substituted alkyl, aryl and heteroaryl. R₂ can be randomly substituted by 1 to 5 substituents selected from the group consisting of halogen, C₁-C₈ alkyl, C₁-C₈ alkoxy, nitryl, cyano group, and trifluoromethyl. More specifically, R₂ can be randomly substituted by 1 to 5 substituents selected from the group consisting of halogen, such as F, Cl, Br, I; a C₁-C₈ alkyl, such as methyl, ethyl, propyl, t-butyl; a C₁-C₈ alkoxy, such as methoxy, ethoxy, propoxy, t-butoxy; nitryl, cyano group, trifluoromethyl.

In other embodiments, R₁ can be n-butyl, benzyl; R₂ can be a randomly substituted alkyl, aryl and heteroaryl. R₂ can be randomly substituted by 1 to 5 substituents selected from the group consisting of halogen, C₁-C₈ alkyl, C₁-C₈ alkoxy, nitryl, cyano group, and trifluoromethyl. Among them, halogen can be F, Cl, Br, I; C₁-C₈ alkyl can be methyl, ethyl, propyl, t-butyl, C₁-C₈ alkoxy can be methoxy, ethoxy, propoxy, t-butoxy.

In other embodiments, R₂ can be C₁-C₄ alkyl, C₆-C₁₀ aryl and C₆-C₁₀ heteroaryl randomly substituted by 1 to 5 substituents selected from methyl, ethyl, propyl, tert-butyl, methoxy, ethoxy, propoxy, tert-butylene, nitryl, cyano group, trifluoromethyl.

More preferably, R₂ is a phenyl randomly substituted by 1 to 5 substituents selected from methyl, methoxy, nitryl, cyano group, trifluoromethyl.

More preferably, R₂ is a phenyl randomly substituted by 1 to 5 substituents selected from methoxyl and trifluoromethyl.

In other embodiments, R₁ can be n-butyl, t-butyl, phenyl, benzyl; R₂ can be C₁-C₄ alkyl, C₆-C₁₀ aryl and C₆-C₁₀ heteroaryl randomly substituted by 1 to 5 substituents selected from methyl, methoxyl, nitryl, cyano group, trifluoromethyl.

In other embodiments, R₁ can be n-butyl, benzyl; R₂ can be phenyl randomly substituted by 1 to 5 substituents selected from methoxyl and trifluoromethyl.

Moreover, the present invention also provides a preparation method of a hydrocarbyl tin complex, the hydrocarbyl tin complex has the following structure:

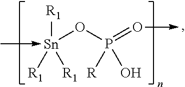

wherein, R₁ is selected from n-butyl, t-butyl, phenyl, benzyl, p-chlorobenzyl and dimethylbenzyl;

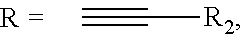

and R₂ is a randomly substituted alkyl, aryl and heteroaryl; n is an integer from 2.

the method comprises the following steps:

in a round-bottom flask, adding tin chloride substituted with R₁ and organic solvent were added, the tin chloride substituted with R₁ was completely dissolved with stirring, then acetylenyl phosphate monosodium substituted with R₂ was added. Reaction was performed under stirring, then the reaction solution was cooled and filtered, the filtrate was concentrated under reduced pressure, and an appropriate amount of petroleum ether was added into the concentrated filtrate, after filtering, vacuum concentration was performed to obtain a product.

In some embodiments, R₁ of the hydrocarbyl tin complex prepared by the method can be n-butyl, t-butyl, phenyl, benzyl; R₂ can be a randomly substituted alkyl, aryl and heteroaryl. R₂ can be randomly substituted by 1 to 5 substituents selected from the group consisting of halogen, C₁-C₈ alkyl, C₁-C₈ alkoxy, nitryl, cyano group, and trifluoromethyl. Among them, halogen can be F, Cl, Br, I; C₁-C₈ alkyl can be methyl, ethyl, propyl, t-butyl, C₁-C₈ alkoxy can be methoxy, ethoxy, propoxy, t-butoxy.

In other embodiments, R₂ can be C₁-C₄ alkyl, C₆-C₁₀ aryl and C₆-C₁₀ heteroaryl randomly substituted by 1 to 5 substituents selected from methyl, ethyl, propyl, tert-butyl, methoxy, ethoxy, propoxy, tert-butylene, nitryl, cyano group, trifluoromethyl.

More preferably, R₁ can be n-butyl, benzyl; R₂ can be phenyl randomly substituted by 1 to 5 substituents selected from methoxyl and trifluoromethyl.

### Embodiment 1: The Synthesis of Tri-n-butyltin (IV) (4-methoxyphenyl) Acetylenyl Phosphonic Acid Derivatives

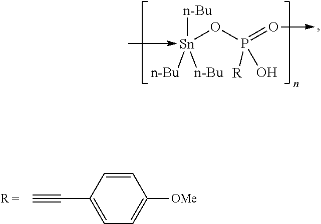

The structural formula of the product is shown as above:

In a 100 ml round-bottom flask, 3 mmol of tri-n-butyl tin chloride and 20 ml of anhydrous methanol were added. The tri-n-butyl tin chloride was completely dissolved with stirring. After that, 3.2 mmol of (4-methoxyphenyl) acetylenyl phosphate monosodium was added. Reaction of the mixture in the flask was held for 4.5 hours under reflux with stirring, then cooled and the filtrate of the reaction solution was concentrated under reduced pressure. An appropriate amount of petroleum ether was added into the concentrated filtrate. After filtering, vacuum concentration was performed to obtain the product, wherein the yield is 87%.

Element Analysis Data: Anal. Calcd. For C₂₁H₃₅O₄PSn (%): C, 53.33, H, 7.04, Sn, 23.69; Found (%): C, 53.37, H, 7.06, Sn, 23.73. Nuclear Magnetic Resonance Spectroscopy Data (¹H NMR/δ): 0.79-0.83 (9H), 1.24-2.03 (18H), 3.42-4.02 (3H), 7.33-7.90 (4H).

### Embodiment 2: The Synthesis of Tri-n-butyltin (IV) (3-Trifluoromethyl Phenyl) Acetylenyl Phosphonic Acid Derivatives

In a round-bottom flask, tri-n-butyl tin chloride and anhydrous methanol were added. The tri-n-butyl tin chloride was completely dissolved with stirring. After that, (3-trifluoromethyl phenyl) acetylenyl phosphate monosodium was added, Reaction of the mixture in the flask was held for 4.5 hours under reflux with stirring, then cooled and the filtrate of the reaction solution was concentrated under reduced pressure. An appropriate amount of petroleum ether was added into the concentrated filtrate. After filtering, vacuum concentration was performed to obtain the product.

### Embodiment 3: Antitumor Activity Test of the Compounds

The compound produced in embodiment 1 is named as “Compound 1”, and the compound produced in embodiment 2 is named as “Compound 2”.

MCF-7, HT-29, A549 and HepG2 cells are got from American Tissue Culture Collection, which are cultured with the culture medium containing 10% bovine fetal serum in a CO₂-containing incubator at 37° C. The MTT method is used to detect cell proliferation and growth inhibition. The number of experimental cells is adjusted to obtain the absorbance at 570 nm, 6 concentrations are set for the compound test solution (0.1 nmol/L˜10 μmol/L). The cells are treated for 72 hours. At least 3 parallel experiments and 3 repeated experiments are conducted for each concentration. The IC₅₀ value is determined by statistical analysis.

The inventors of this application make appropriate structural improvement and screening from existing hydrocarbyl tin complex of alkynyl phosphonic acid on the basis of existing literature, so as to explore new antitumor drugs. According to the preliminary biological activity test, it shows that this kind of hydrocarbyl tin complex of alkynyl phosphonic acid do possess antitumor activity. Among them, several specially-structured complexes especially show excellent activity.

Taking cisplatin as the contrast, the in-vitro growth inhibitory activity of Compound 1 and Compound 2 on tumor cells are tested, including MCF7 (human breast cancer cells), HT-29 (human colon cancer cells), A549 (human lung cancer cells) and HepG2 (liver cancer cells). The results are shown in the table below. It is found that the compounds show stronger antitumor activity than cisplatin, especially for the inhibition of HT-29, and can be used as candidate anticancer compounds.

TABLE 1

| | IC₅₀ Value (μmol/L) | | | |
|---|---|---|---|---|
| Compound | MCF7 | HT-29 | A549 | HepG2 |
| 1 | 0.853 | 0.065 | 0.243 | 0.329 |
| 2 | 1.732 | 0.962 | 0.274 | 0.872 |
| Cisplatin | 43 | 35 | 1.3 | 17 |

We claim:

1. A hydrocarbyl tin complex with antitumor activity, characterized by having the following structure: 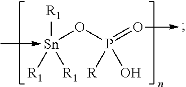
wherein, R₁ is selected from the group consisting of n-butyl, t-butyl, phenyl, benzyl, p-chlorobenzyl and dimethylbenzyl; 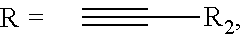
and R₂ is a randomly substituted alkyl, aryl and heteroaryl; n is an integer from 2.

2. The hydrocarbyl tin complex according to claim 1, wherein,
R₂ is a randomly substituted C1-C4 alkyl, C6-C10 aryl and C6-C10 heteroaryl.

3. The hydrocarbyl tin complex according to claim 1, wherein, R₂ is a randomly substituted phenyl.

4. The hydrocarbyl tin complex according to claim 1, wherein,
R₂ is randomly substituted by 1 to 5 substituents selected from the group consisting of halogen, C1-C8 alkyl, C1-C8 alkoxy, nitryl, cyano group, and trifluoromethyl.

5. The hydrocarbyl tin complex according to claim 1, wherein,
R₂ is randomly substituted by 1 to 5 substituents selected from C1-C8 alkoxy, trifluoromethyl.

6. The hydrocarbyl tin complex according to claim 1, wherein,
R₂ is randomly substituted by 1 to 5 substituents selected from methoxyl, trifluoromethyl.

7. The hydrocarbyl tin complex according to claim 3, wherein,
R₂ is randomly substituted by 1 to 5 substituents selected from methoxyl, trifluoromethyl.

8. The hydrocarbyl tin complex according to claim 7, wherein, R₁ is selected from n-butyl and benzyl.

9. A preparation method of a hydrocarbyl tin complex, wherein,
the hydrocarbyl tin complex having the following structure: 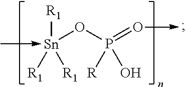
wherein, R₁ is selected from the group consisting of n-butyl, t-butyl, phenyl, benzyl, p-chlorobenzyl and dimethylbenzyl; 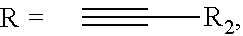
and R₂ is a randomly substituted alkyl, aryl and heteroaryl; n is an integer from 2;
the method comprises:
in a round-bottom flask, adding tin chloride substituted with R₁ and organic solvent, dissolving the tin chloride substituted with R₁ completely with stirring, and adding acetylenyl phosphate monosodium substituted with R₂, performing an reaction under stirring, then cooling and filtering, concentrating the filtrate under reduced pressure, adding an appropriate amount of petroleum ether into the concentrated filtrate, filtering and performing vacuum concentration to obtain a product.

10. The preparation method of a hydrocarbyl tin complex according to claim 9, characterized in that, the hydrocarbyl tin complex of alkynyl phosphonic acid according to claim 1, wherein, R₂ is a randomly substituted C1-C4 alkyl, C6-C10 aryl and C6-C10 heteroaryl.

11. The preparation method of a hydrocarbyl tin complex according to claim 9, characterized in that, R₂ is a randomly substituted phenyl.

12. The preparation method of a hydrocarbyl tin complex according to claim 9, wherein, R₂ is randomly substituted by 1 to 5 substituents selected from C1-C8 alkoxy, trifluoromethyl.

13. The preparation method of a hydrocarbyl tin complex according to claim 9, wherein, R₂ is randomly substituted by 1 to 5 substituents selected from methoxyl, trifluoromethyl.

14. The preparation method of a hydrocarbyl tin complex according to claim 9, wherein, R₁ is selected from n-butyl and benzyl.

15. The preparation method of a hydrocarbyl tin complex according to claim 10, wherein, R₁ is selected from n-butyl and benzyl.

16. The preparation method of a hydrocarbyl tin complex according to claim 11, wherein, R₁ is selected from n-butyl and benzyl.

17. The preparation method of a hydrocarbyl tin complex according to claim 12, wherein, R₁ is selected from n-butyl and benzyl.

18. The preparation method of a hydrocarbyl tin complex according to claim 9, wherein, R₁ is n-butyl and R₂ is a phenyl substituted by trifluoromethyl;
the method comprises:
in a round-bottom flask, adding tri-n-butyl tin chloride and anhydrous methanol, dissolving the tri-n-butyl tin chloride completely with stirring, adding (3-trifluoromethyl phenyl) acetylenyl phosphate monosodium, performing an reaction under reflux with stirring for 4.5 hours, then cooling and filtering, concentrating the filtrate under reduced pressure, adding an appropriate amount of petroleum ether into the concentrated filtrate, filtering and performing vacuum concentration to obtain a product.

19. A preparation method of a hydrocarbyl tin complex, wherein,
the hydrocarbyl tin complex having the following structure: 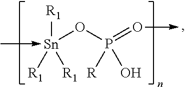
wherein, R₁ is n-butyl, 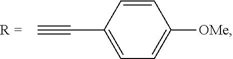
n is an integer from 2;
the method comprises:
in a round-bottom flask, adding tri-n-butyl tin chloride and organic solvent, dissolving the tri-n-butyl tin chloride completely with stirring, and adding (4-methoxyphenyl) acetylenyl phosphate monosodium, performing an reaction under stirring, then cooling and filtering, concentrating the filtrate under reduced pressure, adding an appropriate amount of petroleum ether into the concentrated filtrate, filtering and performing vacuum concentration to obtain a product.

20. The preparation method of a hydrocarbyl tin complex according to claim 19, characterized in that the method comprises: in a 100 ml round-bottom flask, adding 3 mmol of tri-n-butyl tin chloride and 20 ml of anhydrous methanol, dissolving the tri-n-butyl tin chloride completely with stirring, adding 3.2 mmol of (4-methoxyphenyl) acetylenyl phosphate monosodium, performing an reaction under reflux with stirring for 4.5 hours, then cooling and filtering, concentrating the filtrate under reduced pressure, adding an appropriate amount of petroleum ether into the concentrated filtrate, filtering and performing vacuum concentration to obtain a product.
